(19) United States
(12) Patent Application Publication
(10) Pub. No.: US 2008/0260705 A1
(43) Pub. Date: Oct. 23, 2008

(54) Preventing Arrhythmias Associated with Cell Transplantation

(75) Inventors: Eduardo Marban, Beverly Hills, CA (US); Maria Roselle Abraham, Baltimore, MD (US)

Correspondence Address: BANNER & WITCOFF, LTD., 1100 13th STREET, N.W., SUITE 1200, WASHINGTON, DC, 20005-4051, US

(73) Assignee: The Johns Hopkins University, Baltimore, MD (US)

(21) Appl. No.: 10/593,814

(22) PCT Filed: Mar. 22, 2005
(86) PCT No.: PCT/US05/09358

§ 371 Date: Sep. 21, 2007

Related U.S. Application Data
(60) Provisional application No. 60/555,125, filed on Mar. 22, 2004.

(51) Int. Cl. A61K 48/00; G01N 33/483; C12N 5/10; G01N 27/26

(52) U.S. Cl. 424/93.21; 204/403.01; 205/792; 435/375

(57) ABSTRACT

Skeletal myoblasts are an attractive cell type for transplantation since they are autologous and resistant to ischemia. However, clinical trials of myoblasts transplantation in heart failure have been plagued by ventricular tachy-arrhythmias and sudden cardiac death. The pathogenesis of these arrhythmias is poorly understood, but may be related to the fact that skeletal muscle cells, unlike heart cells, are electrically isolated by the absence of gap junctions. An in vitro model of myoblasts transplantation into cardiomyocyte monolayers can be used to investigate the mechanisms of transplant-associated arrhythmias. Co-cultures of human skeletal myoblasts and rat cardiomyocytes result in reentrant arrhythmias (spiral waves) that reproduce the features of ventricular tachycardia seen in patients receiving myoblasts transplants. These arrhythmias can be terminated by nitrendipine, an L-type calcium channel Mocker, but not by the Na channel blocker lidocaine. Genetic modification of myoblasts to stably express the gap junction protein connexin 43 decreases arrhythmogenicity in co-cultures. It similarly can be used to increase the safety of myoblasts transplantation in patients.

This application claims the benefit of provisional applications Ser. Nos. 60/555,125 filed, Mar. 22, 2004, the disclosure of which is expressly incorporated herein.

### TECHNICAL FIELD OF THE INVENTION

This invention is related to the area of cell transplantation. In particular, it relates to transplantation into organs that are contractile or electrically responsive.

### BACKGROUND OF THE INVENTION

Congestive heart failure is a major public health problem in the United States.¹ Cellular myoplasty represents a novel therapy for congestive heart failure, but is fraught with potential pitfalls. Skeletal myoblasts (SkM) are attractive donor cells for myoplasty: they have a contractile phenotype, can be harvested for autologous transplantation, and are resistant to ischemia.² In ongoing phase 2 clinical trials, SkMs are harvested from individual patients via muscle biopsy, grown in culture for 2-4 weeks, and then transplanted by injection into the heart.^{3;4} Despite reports of improvement of contractile indices following myoblast transplantation³⁻⁵, enthusiasm has been tempered by their pro-arrhythmic effects.^{3;4} In the current literature, 10 of the first 22 patients to undergo autologous SkM cardiomyoplasty experienced subsequent ventricular tachycardia or sudden cardiac death.^{3;4} Currently, some myoblast transplantation protocols require administration of the potentially toxic antiarrhythmic drug amiodarone, and placement of an implantable cardioverter defibrillator (ICD) prior to SkM transplantation.⁶

The mechanisms of ventricular arrhythmias associated with SkM cardiomyoplasty remain unknown. Reproducible arrhythmias were not reported in early animal studies (rat⁷⁻⁹, rabbit⁵), and there have been no reports of in vitro models of SkM arrhythmogenesis. Recently, Taylor et al reported more frequent and polymorphic premature ventricular contractions, couplets, triplets, longer pauses following premature atrial contractions and bradycardic death (but not sustained ventricular tachycardia or ventricular fibrillation) following injection of myoblasts in the infarct border zone compared to central scar injection in a rabbit model.¹⁰ Another study of myoblast injection post-infarct did not yield a statistically-significant difference in the incidence of ventricular tachycardia or death between dogs receiving myoblast injections versus saline injections, possibly due to a high frequency of arrhythmias in both groups.¹¹ Hence, in order to pinpoint the role of SkM transplantation in arrhythmogenesis, we designed an in vitro model of myoblast transplantation.

Myoblasts differentiate into myotubes upon injection into the heart.^{7-9;12} Myotubes have very brief action potential duration (APD)⁷ and lack gap junctions and are therefore not coupled to surrounding ventricular myocytes, or to each other.^{7;9} In contrast, cardiomyocytes normally express high levels of the gap junction protein connexin 43 (Cx43), resulting in very efficient electrical coupling of the cardiac syncytium. Hence, we hypothesized that a mixture of myoblasts and myocytes would result in slowing of conduction velocity and greatly increase tissue heterogeneities. Such inhomogeneities predispose to wave-breaks and reentry, key elements of ventricular arrhythmias. Reentry occurs when an impulse fails to die out after normal activation and persists to re-excite the heart.¹³ During reentry, the excitation wave may acquire the shape of an archimedean spiral and is called a spiral wave. Most life-threatening ventricular arrhythmias result from reentrant activity.¹⁴

There is a continuing need in the art for an in vitro model of ventricular tachyeardia. There is also a continuing need in the art for methods of treating diseased hearts and other contractile or electrically responsive organs.

### SUMMARY OF THE INVENTION

One embodiment of the invention is an assay system for simulating cardiac arrhythmias. The assay system comprises a monolayer, co-culture of cardiac myocytes and skeletal muscle myoblasts (SkMM). In addition, it comprises a means for measuring electrical coupling of cells.

Another embodiment of the invention is a method of assaying arrhythmias in cardiac cells in vitro. An electrical property of a monolayer, co-culture of cardiac myocytes and skeletal muscle myoblasts (SkMM) is measured.

Another aspect of the invention is a method of treating myoblasts. A lentivirus encoding a connexin is administered to the myoblasts. The connexin is thereby expressed in the myoblasts.

According to another aspect of the invention a method is provided for treating myoblasts. A nucleic acid encoding a connexin is administered to the myoblasts. The connexin is thereby expressed in the myoblasts. The myoblasts are then transplanted into an organ of a recipient host mammal which is responsive to electrical stimulation.

Yet another aspect of the invention is another method of treating myoblasts. A nucleic acid encoding a calcium channel subunit or a Na-calcium exchanger (NCX) is administered to the myoblasts. The calcium channel subunit or NCX is thereby expressed in the myoblasts. The myoblasts are transplanted into an organ of a recipient host mammal which is responsive to electrical stimulation.

Still another aspect of the invention provides another method of treating myoblasts. A nucleic acid encoding a short hairpin RNA that mimics the structure of an siRNA for a potassium channel is administered to myoblasts. The short hairpin RNA comprises two complementary sequences of 19-21 nucleotides separated by a 5-7 nucleotide spacer region which forms a loop between the two complementary sequences. The short hairpin RNA is expressed in the myoblasts. The myoblasts are transplanted into an organ of a recipient host mammal which is responsive to electrical stimulation.

An additional embodiment of the invention provides a method of treating cells for use in cell transplantation. A lentivirus encoding a connexin is administered to the cells. The connexin is thereby expressed in the cells.

These and other embodiments which will be apparent to those of skill in the art upon reading the specification provide the art with assay systems and methods for assessing and improving electrical conductivity between cells of an electrically responsive and/or contractile organ.

### BRIEF DESCRIPTION OF THE DRAWINGS

FIG. 1A-1D: Myoblast-myocyte signal propagation. (FIG. 1A) Optical action potentials and (FIG. 1B) voltage maps during 2 Hz pacing of myoblast-myocyte co-cultures plated with myocytes on the top half and myoblasts on the bottom half show conduction block at the SkM:NRVM interface. (FIG. 1C) Fluorescent microscopy images (GFP positive myoblasts and myocytes stained red) and (FIG. 1D) calcium transient recordings of myoblast-myocyte co-cultures show lack of propagation of calcium transients from myocytes to neighboring myotubes.

FIG. 2A-2B: Imaging of myoblast-myocyte co-cultures. (FIG. 2A) Transmitted light image of a 1:4 myoblast-myocyte co-culture shows a confluent monolayer. (FIG. 2B) Fluorescent image of Lv-GFP transduced SkM in co-culture with NRVMs in ratio of 1:4 shows a random irregular distribution of myotubes.

FIG. 3A-3C: Impulse propagation. Voltage maps and optical action potentials during propagation of an impulse 50 ms after the stimulus in (FIG. 3A) an NRVM-only monolayer (control, n=7) and (FIG. 3B) a 1:4 Lv-GFP co-culture (n=6). The propagation wavefront is irregular in the co-culture, and propagation is very delayed compared to control. (The color bar in the figure corresponds to normalized voltage level, with blue being the resting state and red being peak of action potential.) Bar graphs display (FIG. 3C) conduction velocity and (FIG. 3D) APD80 (action potential duration at 80% of repolarization) in NRVM-only controls and 1:4 LvGFP co-cultures. Conduction velocity is significantly decreased, while APD80 is significantly increased in co-cultures containing Lv-GFP-transduced myoblasts compared to controls.

FIG. 4: Action potentials from NRVMs in coculture with SkMs. Note the apparent early after depolarizations (arrows).

FIG. 5A-5B: Patterns of reentry. Voltage maps during reentry in two 1:4 Lv-GFP:NRVM co-culture showing (FIG. 5A) single spiral and (FIG. 5B) figure-of-8 spiral. (The color bar in the figure is the same as in FIG. 3A-3B.)

FIG. 6A-6B: Overexpression of Cx43 in myoblasts. (FIG. 6A) Western blot analysis of Cx43 and calsequestrin expression in ventricular myocytes (control), Lv-Cx43-expressing myoblasts and Lv-GFP-expressing myoblasts. (FIG. 6B) Fluorescent images of Cx43 expression in Cx43-transduced myoblasts.

FIG. 7A-7B: Changes in conduction characteristics with Cx43 overexpression. Bar graphs demonstrating (FIG. 7A) conduction velocity and (FIG. 7B) APD80 in 1:4 LvGFP (n=6) and 1:4 LvCx43 (n=−6) co-cultures. Conduction velocity is significantly increased (p<0.01) in Cx43 compared to GFP co-cultures. Additionally, APD80 is significantly decreased (β-0.02) in co-cultures containing Lv-Cx43-transduced myoblasts.

### DETERMINED DESCRIPTION OF THE INVENTION

The inventors have developed an experimental model for arrhythmogenicity of Skeletal myoblast (SkM) transplantation and demonstrate that myoblast-myocyte interactions alone can provide the electrophysiologic milieu for reentrant arrhythmias. These findings explain the clinical observations of high rates of ventricular tachycardia in patients who have undergone autologous SkM transplant following myocardial infarction. Using this model, the inventors have further demonstrated that reentrant arrhythmias can be reduced by transfecting transplanted cells with nucleic acids which encode products that enhance the electrical connections between cells or prolong action potentials.

The assay system of the present invention employs a monolayer co-culture of cardiac myocytes and skeletal muscle myoblasts. The two types of cells can be in adjacent regions or they can be mixed in the same region. A means for measuring electrical coupling of the cells is employed. Electrical coupling can be measured using a voltage-sensitive dye, such as di-4-ANEPPs or di-8-ANEPPS (Molecular Probes) or NK2761, NK2776, NK3224, NK3225, NK3630 (Nippon Kankoh Shikiso Kenkyu-sho) or RH795 (Mo Bi Tec), a fluorescent calcium imaging agent, such as indo-1, acetoxymethyl ester, a calcium ion indicator, such as Rhod-2-AM, a patch clamp apparatus, by measuring conduction velocity or by measuring action potential. Reentrant arrhythmias can be induced by a premature stimulus after pacing or may occur spontaneously.

Cell cultures can be grown on any convenient surface, including glass and plastic. The shape of the surface can be any which is convenient, for example for illumination and recording of emitted light. The surface may be pretreated to enhance adherence of the cells to the surface. Suitable agents for enhancement of adherence include laminin, fibronectin, and collagen. See Entcheva et al., IEEE Transactions on Biomecial Engineering 51:333-341, 2004; Entcheva, et al., J. Cardiovasc. Electrophysiol. 11:665-676, 2000; and Lu et al., Proceedings of IEEE Engineering in Medicine and Biology Society and BMES Annual Conference, Atlanta, October 1999.

The myocytes and myoblasts which are used in the assay system can be from any mammal. They can be, for example, from rodent, ungulate, or primate. They can be from rat, rabbit, mouse, human, cow, pig, dog, or any other suitable source. Adult, embryonic, neonatal, or stem cells can be used. They can be from the same individual animal or from different animals. They can be from the same species source or from different species sources.

Any of various electrical properties can be measured in the assay system. The conduction velocity, transmembrane potential, intracellular calcium, or action potential duration can be measured. These parameters are known in the art and can be measured in the conventional ways.

Polynucleotides encoding a protein for improving the electrical properties of cells delivered by cellular transplantation, such as cellular myoplasty, can be any connexin, in particular connexins 43, 40, 26, 36, 45 and 37. In humans, approximately nine connexins have been identified, and any of these can be used. See, e.g., NM_{—}000165 and NP_{—}00156 (connexin 43), and NM_{—}181703 and NP_{—}859054 (connexin 40) in the NCBI, the sequences as they exist on Mar. 22, 2005, are incorporated by reference herein. Although particular sequences are referenced here, it is accepted that minor variants of up to 1, 2, 3, 4, or 5% of the sequence could be used with the same effect. Connexins improve the electrical conductivity of cells. Proteins other than connexins can be used to improve the electrical properties of cells to be transplanted. For example, calcium channel subunits can be used. A sodium-calcium exchanger (NCX) can also be used. It is also known as SLC8Al (solute carrier family 8) (sodium/calcium exchanger), member 1 [Homo sapiens] and HGNC:11068, NCX1. It has been mapped to human chromosome 2p23-p22. and has a GeneID of 6546. In humans approximately 64 calcium channel subunits have been identified, and any of these can be used. Conversely, it may be desirable to provide a polynucleotide to cells to be transplanted which will make a product, such as antisense RNA, a double-stranded silencing RNA, or a dominant-negative construct, which will inhibit the expression of potassium channels. Approximately 164 potassium channels proteins are known which can be used to design the antisense RNA or silencing RNA, and which can be their targets. These, too, prolong the action potential.

Polynucleotides can be delivered to cells to be transplanted using any suitable vector, including viral vectors or non-viral vectors. Vectors which stably transfect host cells are desirable for generating a long-lasting effect. Lentivirus vectors are one example of a type of vector which can be used to transform cells to be transplanted. Other viruses and plasmid vectors can be used as desired. The effect of the polynucleotides in a particular cell can be confirmed in an assay system as described above. Cell types which can be transfected with polynucleotides include myoblasts, such as skeletal muscle, cardiac muscle, and uterine muscle myoblasts. Other cell types which can be transfected are cardiac stem cells, fibroblasts, and mesenchymal stem cells.

Transplantation of treated myoblasts or other cell types can be accomplished by direct injection into the desired organ. In particular the cells can be directly injected to a site of localized injury. For example cells can be delivered to an infarcted area of a heart or brain. Injection may be by direct visualization, by indirect visualization (e.g., echocardiography-guided needle injection) or by catheter-mediated injection (e.g., under fluoroscopy).

Injection of SkMs into the infarct border zone (characterized by fibrosis²², gap junction remodeling²³ and slow conduction²⁴) would be expected to further slow conduction, promote wave-breaks, and result in an increased risk of reentrant rhythms. Since improvement in function appears to be independent of electrical integration, based on our findings, SkM injection into scar and not the border zone could potentially prevent occurrence of arrhythmias. Cx43 transduction of myoblasts and I_{CaL} blockers could be useful adjuncts in myoblast transplantation to reduce arrhythmias.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Materials and Methods

### Lentivirus

The lenti-vectors pLV-CAG-GFP and pLV-CAG-Cx43-GFP were generated from second generation lentiviral vector, pLV-CAG SIN-18 (Trono lab) under the control of the promoter CAG. Recombinant lentiviruses were generated by co-transfecting HEK293T cells with the plasmids pLV-CAG-GFP or pLV-CAG-Cx43-GFP, pMD.G and pCMVAR8.91 using Lipofectamine 2000 (Invitrogen). Lentiviral particles were harvested at 24 and 48 hrs post-transfection and titered by FACS analysis. For transduction, lentiviruses were added to the myoblasts (MOI=10), with 8 μg/ml polybrene to facilitate transduction. Lentiviral transduction was confirmed by examining GEP expression under fluorescence microscopy (Nikon) and by immunostaining and western blot for Cx43.

### Immunostaining

Cells were fixed with 4% paraformaldehyde for 5 min at room temperature and then permeabilised with 0.075% saponin. Cx43 was detected using a monoclonal mouse anti-Cx43 antibody (Chemicon) and an Alexa Fluor-conjugated secondary antibody. Images were recorded using a two-photon laser scanning microscope (Bio-Rad MRC-1024 MP) with excitation at 740 nm (Tsunami Ti:Sa laser, Spectra Physics). The red emission was collected at 605±25 nm and the green emission 525±25 nm. Images were analyzed offline using ImageJ software (Wayne Rasband, National Institutes of Health) with customized plugins.

### Western Blot

Cells were lysed for 30 mins on ice in lysis buffer (6M Urea, 1% SDS, 20 mM Tris, 1:1000 protease inhibitor (Sigma), 0.1 mM PMSF) and then centrifuged for 10 min at 4,000 rpm. Equivalent samples (5 μg of protein, confirmed by co-probing for Calsequestrin) were loaded for gel electrophoresis on 10% PAGE. After transfer to nitrocellulose, membranes were blocked and probed overnight at 4° C. with primary antibodies for Cx43 (Chemicon Intl, 1:500 dilution). Membranes were incubated with horseradish peroxidase-conjugated secondary antibodies (Amersham Biosciences, UK, 1:1,000 dilution) for 1 hour at room temperature. Protein levels were detected by chemiluminescence and auto-radiography.

### Calcium Transient Imaging

NRVMs and SkM were cultured on 35-mm glass bottom microwell dishes (MatTEK Corp.) for 7 days. Cultures with spontaneous beating were used for calcium transient imaging. Cells were incubated with 3 μM Rhod-2 AM (Molecular Probes) for 30 min at 37° C. The cells were then washed three times and the medium was replaced, after which they were incubated for an additional 60 mins at 37° C. to allow de-esterification of the Rhod-2. Isoproterenol 10 nM was added prior to imaging. Fluorescence imaging was performed at 37° C. using an inverted fluorescence microscope (TE-2000, Nikon) with a cooled CCD camera attachment (Micro Max, Roper Scientific) using WinView32 acquisition software (Roper Scientific). GFP was imaged with 465-495 nm fluorescence excitation and 515-555 nm emission. Rhod-2 was imaged with 528-553 nm excitation and 578-633 nm emission. Ionomycin, 5 μM (Calbiochem) was added at the end of the experiment to confirm uniform loading of Rhod-2.

### Cell Culture

Human skeletal myoblasts were obtained from Cambrex (Walkersville, Md.) and grown in myoblast basal growth medium (SkBM, Clonetics) containing 10% fetal bovine serum, recombinant human epidermal factor (10 ng/ml), dexamethasone (3 μg/ml), L-glutamine, Gentamicin and Amphotericin-B, at 37° C. and 5% CO₂. (Vials obtained from Cambrex contained 70-80% myoblasts, and the remainder were fibroblasts). The cells were seeded at 3,500 cells/cm² and maintained at cell densities of 60-70% to prevent myotube formation during the culture process. Cells were transduced with lentivirus on their second passage and frozen at −80° C. or amplified up to 10 population doublings. For co-cultures, myoblasts were dissociated using trypsin, counted and then used.

### Cardiac Cells

NRVMs were dissociated from ventricles of 2-day old neonatal Sprague-Dawley rats (Harlan; Indianapolis, Ind.) with the use of trypsin (US Biochemicals; Cleveland Ohio) and collagenase (Worthington; Lakewood, N.J.) as previously described.¹⁵ The investigation conforms to the protocols in the National Institutes of Health Guide for the care and use of animals (NIH publication No. 85-23, Revised 1996). Cells were re-suspended in M199 culture medium (Life Technologies, Rockville, Md.), supplemented with 10% heat-inactivated fetal bovine serum (Life Technologies), differentially pre-plated in two 45 minute steps, and then counted using a hemocytometer. For control experiments, 10⁶ cells were plated on 22 mm plastic coverslips coated with fibronectin (25 μg/ml). On day 2 after cell plating, serum was reduced to 2%.

### Co-Cultures

Myoblasts and NRVMs were co-cultured (isotropic) on 22 mm plastic cover slips (coated with fibronectin, 25 μg/ml) for 9-11 days and then used for optical mapping. In an initial set of experiments, 0.5×10⁶ NRVMs were plated over half of the cover slip, with the other half covered by a PDMS stamp coated with fibronectin (50 μg/ml). The PDMS stamp was removed 24 hours later and 0.5×10⁶ myoblasts transduced with Lv-GFP were then plated. This experiment was performed to ascertain whether or not there is electrical propagation between NRVMs and myotubes. In a second set of experiments, the myoblasts (transduced with LvGFP) and NRVMs were plated at the same time in varying ratios: 1:1, 1:4 and 1:9 to study the electrophysiologic consequences of mixing the two cell types.

On day 2 after cell plating, serum was reduced to 2%. An additional set of experiments (n=3) was performed in 1:4 (non GFP-transduced) myoblast: myocyte co-cultures. Next, myoblasts transduced with Lv-Cx43 were co-cultured with NRVMs in ratios of 1:1 and 1:4.

### Optical Mapping

Coverslips were visually inspected under a microscope. Monolayers with obvious gaps in confluency and non-beating cultures were rejected. The coverslips were placed in a custom-designed chamber, stained with 5 μM di-4-ANEPPS (Molecular Probes; Eugene, Oreg.) for 5 min and continuously superfused with warm (36.5° C.) oxygenated Tyrode solution consisting of (in mM) 135 NaCl, 5.4 KCl, 1.8 CaCl₂, 1 MgCl₂, 0.33 NaH₂PO₄, 5 HEPES, and 5 Glucose. A unipolar point or area electrode (4 bipolar line electrodes) was used to stimulate the cells in culture. Action potentials were recorded from 253 sites using a modified custom-built contact fluorescence imaging system.¹⁵ The recording chamber was placed directly above a fiber bundle with fibers arranged in a 17 mm-diameter hexagonal array. A light emitting diode (LED) light source with an interference filter (530+/−25 mm) delivered excitation light to the chamber. A plexi-glass cover was placed on top of the chamber to stabilize the solution surface and reduce optical artifacts. The bottom of the chamber consisted of a No. 1 circular glass coverslip spin-coated with 3 layers of red ink (Avery Dennison; Brea, Calif.) to attenuate the excitation light and pass the red emission signal. Optical signals were low pass filtered at 500 Hz and amplified with eight custom-designed 32-channel printed circuit boards. Signals were sampled at 1 kHz and digitized with four, 64 channel 16 bit analog-to-digital boards (Sheldon Instruments, San Diego, Calif.). Data was stored, displayed, and analyzed using software written in Visual C++(Microsoft; Redmond, Va.), Lab VIEW (Texas Instruments; Dallas, Tex.) and MATLAB (Math Works; Natick, Mass.).

### Experimental Protocol

A is recording was initially made to check for spontaneous activity. 15 beat drive trains of 10 ms monophasic pulses (1.5× diastolic threshold) were subsequently used for stimulation throughout the experiment. Stimulation was begun at 1 Hz and increased progressively by 1 Hz until 1:1 capture was no longer observed, or reentry was initiated. Nitrendipine (5 μM) or Lidocaine (200 μM) in warm (36.5° C.) Tyrode was superfused into the experimental chamber and 2 sec recordings were obtained every 30-60 sec for 10 min or until termination of reentry. The drug was then washed out over 10 min with Tyrode solution and another recording was obtained. If reentry was terminated, stimulation was begun at 1 Hz and increased as before. If reentry was not terminated or if re-initiated, a second drug was introduced. We constructed a dose response curve with Nitrendipine and found that nitrendipine (5 μM) shortened APD by 50% but did not affect conduction velocity. Higher doses of Nitrendipine produce Na channel blockade in addition to L-type calcium channel blockade.¹⁶

### Data Analysis

Baseline drift was reduced by subtraction of a fitted polynomial curve from the optical signal. Animations of electrical propagation were generated from signals that were band-pass filtered between 0 and 100 Hz. The activation time was defined as the instant of maximum positive slope. Co-cultures with a myoblast:myocyte ratio of 1:4 during the plating step were used for analysis of CV and APD. The relative activation times at each recording point of the hexagonal array were used to calculate conduction velocity. To compare velocities among different episodes in the same monolayer, conduction velocity was calculated along the same path and averaged over different stimulus responses. Paths were chosen to be sufficiently far away from the stimulus site so that latency delays associated with excitation could be neglected. Data are expressed as Mean +/−SEM unless stated otherwise. Differences between means were assessed using the Student's t test or Fischer's exact test.

### Electrophysiology

The action potentials from (non-dissociated) control and co-cultured NRVMs were measured in perforated patches using current-clamp mode with Axopatch 200B (Axon Instruments). The bath solution contained NaCl 140 mM, KCl 4 mM, CaCl₂ 2 mM, MgCl₂ 1 mM, glucose 10 mM, HEPES 10 mM, pH=7.4 with NaOH (normal Tyrode's), and the pipette solution contained K-Aspartate 110 mM, KCl 20 mM, MgCl₂ 1 mM, EGTA 10 mM, MgATP 5 nM, GTP 0.1 mM, Phosphocreatine Na₂ 5 mM, HEPES10 mM, pH=7.3 with KOH, plus 120 μg/mL of nystatin for perforated patch.

### EXAMPLE 2

### Lack of Electrical Coupling Between Adjacent Cultures

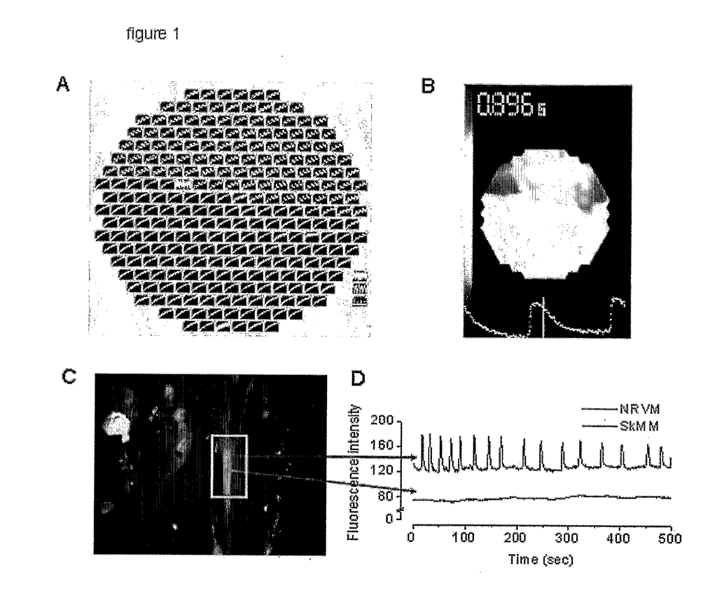

One likely contributor to arrhythmias following myoblast transplantation is the predicted absence of electrical coupling between NRVMs and myotubes. Indeed, mathematical simulations have shown that, with decreased gap junction coupling, conduction is very slow but, paradoxically, very robust (due to an increase in the safety factor for propagation), increasing the tendency for reentry.¹⁷ We confirmed the lack of electrical coupling at a syncytial level by optical mapping of co-cultures plated with SkMs on one half and NRVM on the other half of the coverslip. Stimulation on the NRVM half resulted in a propagated wave-front that blocked at the NRVM/SkM interface (FIG. 1a, b). The absence of electrical coupling was confirmed at a single-cell level by measuring lack of propagation of calcium transients between neighboring myocytes and myotubes using Rhod-2 AM (5 μM) as the calcium indicator. (FIG. 1c, d).

### EXAMPLE 3

### Lack of Electrical Coupling in Mixed Co-Cultures

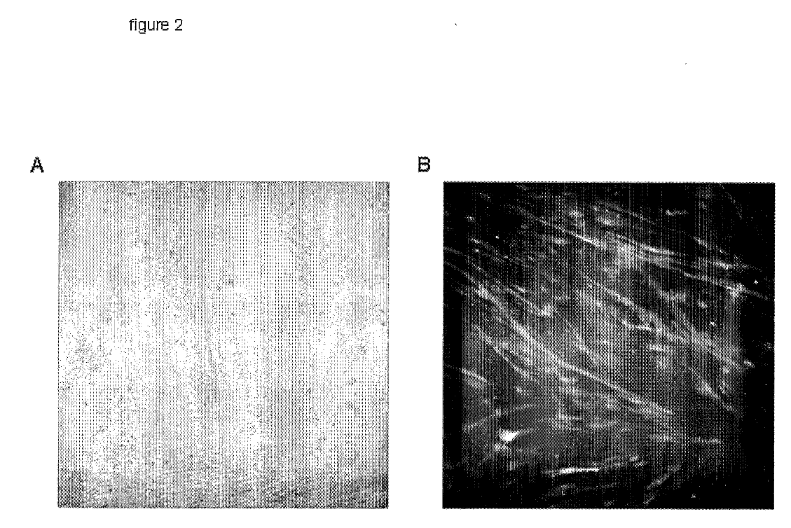

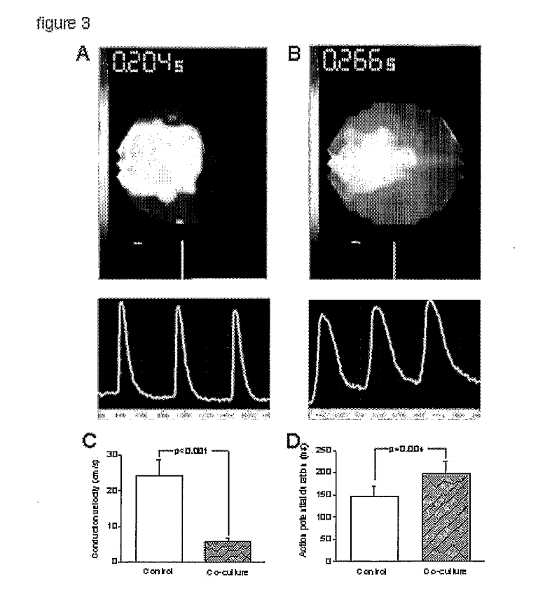

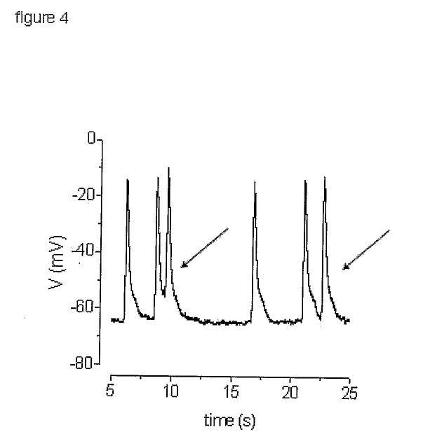

We next proceeded to characterize mixed co-cultures, a situation that mimics the engraftment of SkM in hearts in vivo.⁶ Light (FIG. 2a) and fluorescence microscopy (FIG. 2b) revealed that myotubes tend to grow in linear irregular patterns. The electrically-uncoupled myotubes interspersed among NRVMs would be expected to behave as localized barriers to propagation, resulting in slowing of overall conduction and predisposing to irregularities in the wave-front, source-load mismatch, wave-break and reentry.¹⁸⁻²⁰ Indeed, optical mapping of mixed SkM/NRVM co-cultures revealed greatly decreased conduction velocity in all SkM: NRVM co-cultures, compared to control (NRVM-only) cultures. FIG. 3a, b shows conduction velocity in co-cultures compared to control. Additionally, action potential duration (APD80) in co-cultures was prolonged. This unanticipated delay of cardiac repolarization represents a novel pro-arrhythmic effect²¹ of SkM co-culture, above and beyond the predictable slowing of conduction, and may be due to a paracrine effect of SkMs. In fact, whole cell patch clamp of NRVMs in co-culture, but not in control cultures, revealed evidence of APD prolongation and triggered activity. (FIG. 4)

In co-cultures, (but not in the controls), the depolarization wavefront was irregular, with wave-breaks occurring at pacing rates of 4-6 Hz and preceding reentry initiation. Additionally, lack of 1:1 conduction developed at a pacing rate of 4-6 Hz in co-cultures, but only at a high pacing rate of 8-11 Hz in NRVM controls.

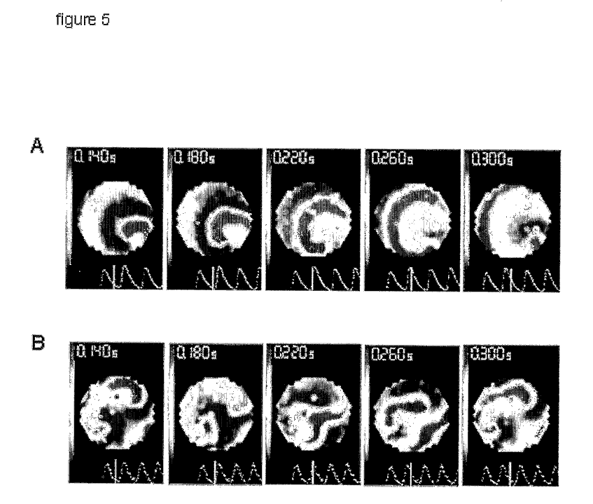

Reentrant rhythms (spiral waves) were easily inducible by rapid pacing in 100% of the mixed co-cultures (n=14; SkM:NRVM ratios of 1:1, 1:4, and 1:9). In contrast, reentry could not be induced in NRVM-only controls. In one 1:4 co-culture, spontaneous reentry was present prior to pacing. The spontaneous and induced reentrant rhythms (FIG. 5a, b) were varied: single, multiple or figure-of-eight (two counter-rotating spirals) spirals that were stable, drifting or transient.

### EXAMPLE 4

### Pharmacological Intervention for Reentry Arrhythmias

Most (90%) of the induced reentrant arrhythmias were sustained for >5 mins, making them amenable to pharmacologic intervention. High-dose lidocaine (200 μM), a Na channel blocker and commonly used anti-arrhythmic, slowed the reentry rate by 70-80% but did not terminate it in the majority of co-cultures (n=12). In contrast, nitrendipine (5 μM), an L-type calcium current (I_{CaL}) blocker, slowed the reentrant rhythms by a modest 10-20% before abrupt termination within 5 min (n=12) in all co-cultures. The observed dependence of propagation On I_{CaL} provides further support for the notion that decreased gap junction coupling underlies the decrease in conduction velocity and inducibility of reentry in co-cultures. In fact, mathematical modeling¹⁷ and experimental data¹⁸ have shown that, with decreased gap junction coupling, conduction delays between cells or groups of cells markedly exceed the rise-time of the action potential upstroke, making propagation increasingly dependent on I_{CaL} rather than Na current.

### EXAMPLE 5

### Genetic Enhancement of Cell Coupling

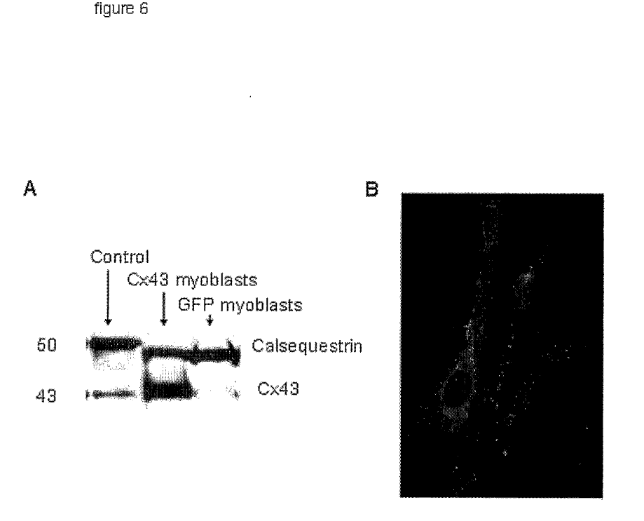

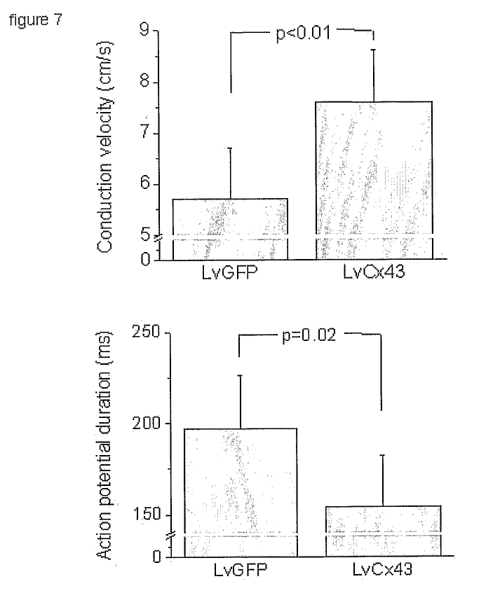

Pharmacotherapy with calcium channel blockers for arrhythmias is limited by side effects such as hypotension and contractile failure. As an alternative means to decrease arrhythmogenesis, we investigated genetic enhancement of cell-cell coupling by stable lentivirally-mediated transduction of SkM with Cx43. Western blot (FIG. 6a) showed greatly increased Cx43 expression compared even to ventricular myocyte controls. Immunostaining (FIG. 6b) revealed plaques in the membrane as well as a large amount of punctate staining in the membrane and in the cytoplasm. In Cx43-expressing SkM-NRVM co-cultures, conduction velocity was increased by 30% and APD80 was decreased by 20% compared to the Lv-GFP co-cultures (FIG. 7a, b). Sustained reentry was induced in only 2 of 9 Cx43-transduced co-cultures compared to 13 of 14 Lv-GFP-transduced co-cultures (p=0.001, Fischer's exact test). These results show that genetic modification of SkM to express Cx43 prior to transplantation protects against arrhythmias in co-cultures. Further in vivo studies are needed to address the role of Cx43 over-expression in myoblast transplantation.

Our results provide the first experimental model for arrhythmogenicity of SkM transplantation and demonstrate that myoblast-myocyte interactions alone can provide the electrophysiologic milieu for reentrant arrhythmias. These findings rationalize the clinical observations of high rates of ventricular tachycardia in patients who have undergone autologous SkM transplant following myocardial infarction. Injection of SkMs into the infarct border zone (characterized by fibrosis²², gap junction remodeling²³ and slow conduction²⁴) would be expected to further slow conduction, promote wave-breaks, and result in an increased risk of reentrant rhythms. Since improvement in function appears to be independent of electrical integration, based on our findings, SkM injection into scar and not the border zone could potentially prevent occurrence of arrhythmias. Cx43 transduction of myoblasts and I_{CaL} blockers could be useful adjuncts in myoblast transplantation to reduce arrhythmias.

### REFERENCES

The disclosure of each reference cited is expressly incorporated herein, in particular for the subject matter described in the text which refers to it.
1. Ho K K, Anderson K M, Kannel W B, Grossman W, Levy D. Survival after the onset of congestive heart failure in Framingham Heart Study subjects. Circulation. 1993; 88: 107-115.
2. Menasche P. Skeletal muscle satellite cell transplantation. Cardiovasc Res. 2003; 58:351-357.
3. Menasche P, Hagege A A, Vilquin J T, Desnos M, Abergel E, Pouzet B, Bel A, Sarateanu S, Scorsin M, Schwartz K, Bruneval P, Benbunan M, Marolleau J P, Duboc D. Autologous skeletal myoblast transplantation for severe postinfarction left ventricular dysfunction. J Am Coll Cardiol. 2003; 41:1078-1083.
4. Smits P C, van Geuns R J, Poldernans D, Bountioukos M, Onderwater E E, Lee C H, Maat A P, Serruys P W. Catheter-based intrainyocardial injection of autologous skeletal myoblasts as a primary treatment of ischemic heart failure: clinical experience with six-month follow-up. J Am Coll Cardiol. 2003; 42:2063-2069.
5. Taylor D A, Atkins B Z, Hungspreugs P, Jones T R, Reedy M C, Hutcheson K A, Glower D D, Kraus W E. Regenerating functional myocardium: improved performance after skeletal myoblast transplantation. Nat. Med. 1998; 4:929-933.
6. Minami E, Reinecke H, Murry C E. Skeletal muscle meets cardiac muscle. Friends or foes? J Am Coll Cardiol. 2003; 41:1084-1086.
7. Leobon B, Garcin I, Menasche P, Vilquin J T, Audinat E, Charpak S. Myoblasts transplanted into rat infarcted myocardium are functionally isolated from their host. Proc Natl Acad Sci USA. 2003; 100:7808-7811.
8. Al Attar N, Canion C, Ghostine S, Garcin I, Vilquin J T, Hagege A A, Menasche P. Long-term (1 year) functional and histological results of autologous skeletal muscle cells transplantation in rat. Cardiovasc Res. 2003; 58:142-148.
9. Murry C E, Wiseman R W Schwartz S M, Hausclika S D. Skeletal myoblast transplantation for repair of myocardial necrosis. J Clin Invest. 1996; 98:2512-2523.
10. Soliman A M, Iucoff M W, Crater S, Morimoto Y, Taylor D A. Cell location may be a primary determinant of safety after myoblast transplantation into the infarcted heart. J Am Coll Cardiol. 2004; 43:15A. (Abstract)
11. Sherman W, He K-L, Yi G-H, Zhou H, Gu A, Becker E M, Zhang G-P, Harvey J, Kao R, Lee M J, Wang J, Haimes H, Burkhoff, D. Ventricular arrhythmias following autologous skeletal myoblast implantation. J Am Coll Cardiol. 2003; 41:176A. (Abstract)
12. Hagege A A, Carrion C, Menasche P, Vilquin J T, Duboc D, Marolleau J P, Desnos M, Bruneval P. Viability and differentiation of autologous skeletal myoblast grafts in ischaemic cardiomyopathy. Lancet. 2003; 361:491-492.
13. Antzelevitch C. Basic mechanisms of reentrant arrhythmias. Curr Opin Cardiol. 2001; 16:1-7.
14. Zipes D P. Mechanisms of clinical arrhythmias. J Cardiovasc Electropllysiol. 2003; 14:902-912.
15. Iravanian S, Nabutovsky Y, Kong C R, Saha S, Bursac N, Tung L. Functional reentry in cultured monolayers of neonatal rat cardiac cells. Am J Physiol Heart Circ Physiol. 2003; 285:H449-H456.
16. Yatani A, Brown A M. The calcium channel blocker nitrendipine blocks sodium channels in neonatal rat cardiac myocytes. Circ Res. 1985; 56:868-875.
17. Shaw R M, Rudy Y. Ionic mechanisms of propagation in cardiac tissue. Roles of the sodium and L-type calcium currents during reduced excitability and decreased gap junction coupling. Circ Res. 1997; 81:727-741.
18. Rohr S, Kucera J P, Kleber A G. Slow conduction in cardiac tissue, I: effects of a reduction of excitability versus a reduction of electrical coupling on microconduction. Circ Res. 1998; 83:781-794.
19. Kucera J P, Kleber A G, Rohr S. Slow conduction in cardiac tissue, II: effects of branching tissue geometry. Circ Res. 1998; 83:795-805.
20. Kleber A G, Rudy Y. Basic mechanisms of cardiac impulse propagation and associated arrhythmias. Physiol Rev. 2004; 84:431-488.
21. Tomaselli G F, Beuckelmann D J, Calkins H G, Berger R D, Kessler P D, Lawrence J H, Kass D, Feldman A M, Marban E. Sudden cardiac death in heart failure. The role of abnormal repolarization. Circulation. 1994; 90:2534-2539.
22. Ursell P C, Gardner P I, Albala A, Fenoglio J J, Jr., Wit A L. Structural and electrophysiological changes in the epicardial border zone of canine myocardial infarcts during infarct healing. Circ Res. 1985; 56:436-451.
23. Smith J H, Green C R, Peters N S, Rothery S, Severs N J. Altered patterns of gap junction distribution in ischemic heart disease. An immunohistochemical study of human myocardium using laser scanning confocal microscopy. Am J Pathol. 1991; 139:801-821.
24. Gardner P I, Ursell P C, Fenoglio J J, Jr., Wit A L. Electrophysiologic and anatomic basis for fractionated electrograms recorded from healed myocardial infarcts. Circulation. 1985; 72:596-611.

We claim:

1. An assay system for simulating cardiac arrhythmias, comprising:
a monolayer, co-culture of cardiac myocytes and skeletal muscle myoblasts (SkMM); and
a means for measuring electrical coupling of cells.

2. The assay system of claim 1 wherein the means comprises a voltage-sensitive dye.

3. The assay system of claim 1 wherein the means comprises voltage-sensitive dye di-4-ANEPPS.

4. The assay system of claim 1 wherein the means comprises fluorescent calcium imaging agent Indo-1, acetoxymethyl ester (indo-1-AM).

5. The assay system of claim 1 wherein the means is a calcium ion indicator.

6. The assay system of claim 1 wherein the means is a patch clamp apparatus.

7. The assay system of claim 1 wherein the means measures conduction velocity.

8. The assay system of claim 1 wherein the means measures action potential duration.

9. The assay system of claim 5 wherein the means is calcium ion indicator Rhod-2-AM.

10. The assay system of claim 1 further comprising an electrode.

11. The assay system of claim 1 wherein the cardiac myocytes are neonatal myocytes.

12. The assay system of claim 1 wherein the cardiac myocytes are neonatal rat myocytes (NRCM).

13. The assay system of claim 1 wherein the cardiac myocytes are ventricular myocytes.

14. The assay system of claim 1 wherein the cardiac myocytes are neonatal ventricular myocytes.

15. The assay system of claim 1 wherein the cardiac myocytes are neonatal rat ventricular myocytes (NRVM).

16. A method of assaying arrhythmias in cardiac cells in vitro, comprising:
measuring an electrical property of a monolayer, co-culture of cardiac myocytes and skeletal muscle myoblasts (SkMM).

17. The method of claim 16 wherein the step of measuring employs a voltage-sensitive dye.

18. The method of claim 16 wherein the step of measuring employs voltage-sensitive dye di-4-ANEPPS.

19. The method of claim 16 wherein the step of measuring employs fluorescent calcium imaging agent Indo-1, acetoxymethyl ester (indo-1-AM).

20. The method of claim 16 wherein the step of measuring employs a calcium ion indicator.

21. The method of claim 16 wherein the step of measuring employs a patch clamp apparatus.

22. The method of claim 16 wherein the step of measuring determines conduction velocity.

23. The method of claim 16 wherein the step of measuring determines action potential duration.

24. The method of claim 16 wherein the step of measuring employs calcium ion indicator Rhod-2-AM.

25. The method of claim 16 wherein the step of measuring employs an electrode.

26. The method of claim 16 wherein the cardiac myocytes are neonatal myocytes.

27. The method of claim 16 wherein the cardiac myocytes are neonatal rat myocytes (NRCM).

28. The method of claim 16 wherein the cardiac myocytes are ventricular myocytes.

29. The method of claim 16 wherein the cardiac myocytes are neonatal ventricular myocytes.

30. The method of claim 16 wherein the cardiac myocytes are neonatal rat ventricular myocytes (NRVM).

31. A method of treating myoblasts, comprising:
administering to the myoblasts a lentivirus encoding a connexin, whereby the connexin is expressed in the myoblasts.

32. The method of claim 31 wherein the connexin is connexin 43.

33. The method of claim 31 wherein the connexin is connexin 40.

34. The method of claim 31 further comprising the step of transplanting the treated myoblasts into a recipient host mammal.

35. The method of claim 31 further comprising the step of transplanting the treated myoblasts into a recipient host mammal's heart.

36. The method of claim 31 further comprising the step of transplanting the treated myoblasts into a recipient host mammal's brain.

37. The method of claim 31 further comprising the step of transplanting the treated myoblasts into a recipient host mammal's muscle.

38. The method of claim 31 further comprising the step of transplanting the treated myoblasts into a recipient host mammal's uterus.

39. The method of claim 31 wherein the myoblasts are skeletal muscle myoblasts.

40. The method of claim 31 wherein the myoblasts are cardiac muscle myoblasts.

41. The method of claim 31 wherein the myoblasts are uterine muscle myoblasts.

42. The method of claim 34 wherein the myoblasts are autologous to the recipient host mammal.

43. A method of treating myoblasts, comprising:
administering to the myoblasts a nucleic acid encoding a connexin, whereby the connexin is expressed in the myoblasts; and
transplanting the myoblasts into an organ of a recipient host mammal which is responsive to electrical stimulation.

44. The method of claim 43 wherein the connexin is connexin 43.

45. The method of claim 43 wherein the connexin is connexin 40.

46. The method of claim 43 wherein the nucleic acid is a stable vector.

47. The method of claim 43 wherein the myoblasts are stably transfected by the nucleic acid.

48. The method of claim 43 wherein the nucleic acid is a lentivirus vector.

49. The method of claim 43 wherein the organ is a heart.

50. The method of claim 43 wherein the organ is a brain.

51. The method of claim 43 wherein the organ is a muscle.

52. The method of claim 43 wherein the organ is a uterus.

53. The method of claim 43 wherein the myoblasts are skeletal muscle myoblasts.

54. The method of claim 43 wherein the myoblasts are cardiac muscle myoblasts.

55. The method of claim 43 wherein the myoblasts are uterine muscle myoblasts.

56. The method of claim 43 wherein the myoblasts are autologous to the recipient host mammal.

57. A method of treating myoblasts, comprising:
administering to the myoblasts a nucleic acid encoding a calcium channel subunit, whereby the calcium channel subunit is expressed in the myoblasts; and
transplanting the myoblasts into an organ of a recipient host mammal which is responsive to electrical stimulation.

58. The method of claim 43 wherein the calcium channel subunit is an alpha subunit.

59. The method of claim 43 wherein the calcium channel subunit is a beta subunit.

60. The method of claim 43 wherein the nucleic acid is a stable vector.

61. The method of claim 43 wherein the myoblasts are stably transfected by the nucleic acid.

62. The method of claim 43 wherein the nucleic acid is a lentivirus vector.

63. The method of claim 43 wherein the organ is a heart.

64. The method of claim 43 wherein the organ is a brain.

65. The method of claim 43 wherein the organ is a muscle.

66. The method of claim 43 wherein the organ is a uterus.

67. The method of claim 43 wherein the myoblasts are skeletal muscle myoblasts.

68. The method of claim 43 wherein the myoblasts are cardiac muscle myoblasts.

69. The method of claim 43 wherein the myoblasts are uterine muscle myoblasts.

70. The method of claim 43 wherein the myoblasts are autologous to the recipient host mammal.

71. A method of treating myoblasts, comprising:
administering to the myoblasts a nucleic acid encoding a short hairpin silencing RNA (siRNA) for a potassium channel, wherein the short hairpin silencing RNA comprises two complementary sequences of 19-21 nucleotides separated by a 5-7 nucleotide spacer region which forms a loop between the two complementary sequences, whereby the short hairpin RNA is expressed in the myoblasts; and
transplanting the myoblasts into an organ of a recipient host mammal which is responsive to electrical stimulation.

72. The method of claim 43 wherein the potassium channel is voltage-gated channel.

73. The method of claim 43 wherein the potassium channel is cardiac potassium channel.

74. The method of claim 43 wherein the nucleic acid is a stable vector.

75. The method of claim 43 wherein the myoblasts are stably transfected by the nucleic acid.

76. The method of claim 43 wherein the nucleic acid is a lentivirus vector.

77. The method of claim 43 wherein the organ is a heart.

78. The method of claim 43 wherein the organ is a brain.

79. The method of claim 43 wherein the organ is a muscle.

80. The method of claim 43 wherein the organ is a uterus.

81. The method of claim 43 wherein the myoblasts are skeletal muscle myoblasts.

82. The method of claim 43 wherein the myoblasts are cardiac muscle myoblasts.

83. The method of claim 43 wherein the myoblasts are uterine muscle myoblasts.

84. The method of claim 43 wherein the myoblasts are autologous to the recipient host mammal.

85. A method of treating cells for use in cell transplantation, comprising:
administering to the cells a lentivirus encoding a connexin, whereby the connexin is expressed in the cells.

86. The method of claim 85 wherein the cells are selected from the group consisting of fibroblasts, mesenchymal stem cells, and cardiac stem cells.

87. The method of claim 85 wherein the connexin is connexin 43.

88. The method of claim 85 wherein the connexin is connexin 40.

89. The method of claim 85 further comprising the step of transplanting the treated cells into a recipient host mammal.

90. The method of claim 85 further comprising the step of transplanting the treated cells into a recipient host mammal's heart.

91. The method of claim 85 further comprising the step of transplanting the treated cells into a recipient host mammal's brain.

92. The method of claim 85 further comprising the step of transplanting the treated cells into a recipient host mammal's muscle.

93. The method of claim 85 further comprising the step of transplanting the treated cells into a recipient host mammal's uterus.

94. The method of claim 85 wherein the cells are fibroblasts.

95. The method of claim 85 wherein the cells are mesenchymal stem cells.

96. The method of claim 85 wherein the cells are cardiac stem cells.

97. The method of claim 89 wherein the myoblasts are autologous to the recipient host mammal.
